(19) United States
(12) Patent Application Publication
(10) Pub. No.: US 2006/0025842 A1
(43) Pub. Date: Feb. 2, 2006

(54) Low frequency therapy apparatus

(76) Inventor: Toshimasa Mochizuki, Shizuoka (JP)

Correspondence Address: BIRCH STEWART KOLASCH & BIRCH, PO BOX 747, FALLS CHURCH, VA, 22040-0747, US

(21) Appl. No.: 10/902,171

(22) Filed: Jul. 30, 2004

(51) Int. Cl. A61N 1/05

(52) U.S. Cl. 607/145

(57) ABSTRACT

A non-symmetric property spike pulse Ps is set to have a frequency of 8.0±0.5 Hz, a pulse width Tp of a range from 210 μsec to 650 μsec, and also a turns ratio between a primary winding 23f and a secondary winding 23r of an output transformer 23 is set so that a positive peak voltage value Vp of the non-symmetric property spike pulse Ps during the pulse width Tp period becomes from 60 V to 100 V, when being applied at 300 KΩ load which corresponds to a load resistance of a human body, and a positive voltage during the pulse width Tp period is kept to be equal to or more than a predetermined voltage value Vx. The pulse width Tp of the non-symmetric property spike pulse Ps is changeable within the range from 210 μsec to 650 μsec by a setting change circuit 26, and the turns ratio of the output transformer 23 is set to be “the primary winding:the secondary winding” is from “1:14” to “1:22”.

### BACKGROUND OF THE INVENTION

1. Field of the Invention

The present invention relates to a low frequency therapy apparatus for soothing or removing a local pain of a human body by using a non-symmetric property spike pulse.

2. Description of the Related Art

Conventionally, a low frequency therapy apparatus flowing a low frequency current to a local region (affected part) of a human body so as to activate cells by an electric stimulus is known. In this type of low frequency therapy apparatus, the effect of improving diseases by the activation of cells is recognized when it is used properly, but it is not enough as for a therapy of a pain such as soothing or removing the pain. In other words, it does not directly encounter to the pain, but a whole cell is activated successfully, and the pain is soothed by a secondary action thereof.

The present inventor has already proposed a low frequency therapy apparatus preferable for use in soothing or removing a pain by a direct action on the pain (International Publication No. WO02/000295). The low frequency therapy apparatus includes, a spike pulse generating circuit, generating a non-symmetric property spike pulse which is built in a barrel unit, a grip portion, provided on one side on the barrel unit, a plurality of needle-shaped conductors electrically connected to the non-symmetric property spike pulse generating circuit, being protrudingly provided on the other side of the barrel unit, and a ground means, exposed on the surface of the grip portion. In this case, the spike pulse generating circuit generates the low frequency non-symmetric property spike pulse of 8.0±0.2 Hz, and a pulse current of the non-symmetric property spike pulse generated at the spike pulse generating circuit is set to be equal to or less than 0.48±20% mA, and a peak to peak of a pulse voltage is set to be from 320±20% V to 220±20% V. Hereby the non-symmetric property spike pulse current in low frequency flows to the affected part having a pain caused by a muscle ache or the like, from the intensively arranged many needle-shaped conductors. Thereby the ischemic condition of a body tissue is abolished, a production of a new pain producing substance is suppressed, furthermore, an abnormal low frequency comes out of a pain-sensing organ is adjusted, thereby the pain is soothed and removed.

However, the low frequency therapy apparatus still have rooms for further improvement. That is to say, this type of low frequency therapy apparatus performs a therapy by the action of the current flowed through the human body, therefore it is deeply involved in a physiological mechanism of the human body. Generally, it is already made known by a neuroimmunology that a pain passes from peripheral nerves through a spinal cord being a central nerve, reaches a cerebrum and makes it aware of a pain, by a stimulus being a cause of the pain. At this time, when the stimulus is transferred on the level of spinal cord, a motor nerve is excited and a muscle tension is increased. Furthermore, a sympathetic nerve is activated, and a substance called catecholamine is secreted from an adrenal gland. Next, blood vessels shrink and a blood shortage arises in cellular structures. When this abnormal metabolic turnover is continuing, a bradykinin or a serotonine known as pain producing substances are secreted. And a mechanism in which those substances pass from the peripheral nerves through the spinal cord, and the pain is recognized at the cerebrum again, works.

Meanwhile, it is known that the human body adjusts the balance by the activity of nerves and electrical signals of ion, and there exist unique frequencies. For example, a cardiac current from a heart, brain waves from a brain, an actuating current from nerves and muscles, a damaged current from an injured region, and so on arise and it is called as a weak current theory. Concretely speaking, there exists more plus electric charge inside of a cell, and more minus electric charge on the cell membrane outside of a cell, and a good health is maintained by the even ion balance. Generally, the ion balance is maintained by potassium ions and sodium ions, and there are more potassium ions inside of the cell membrane, on the contrary, there are less potassium ions and more sodium ions outside of the cell membrane, and they are adjusted by a cerebrum and information on end of nerve cells. In addition, as for a brain wave, it becomes a beta wave of equal to or more than 13 Hz in a daily life or in a state of tension, it becomes an alpha wave of from 7 Hz to 13 Hz in a proper relaxed state, and it becomes a theta wave of from 4 Hz to 7 Hz in a dazed or sleepy state.

As described above, a physiological mechanism of human body stands on an extremely accurate and delicate balance, thereby in the above-described low frequency therapy apparatus performing a therapy by using an action of the current based on the non-symmetric property spike pulse flowing through a human body, it is not enough just to quantitatively set the physical quantities of the frequency, the voltage, and so on, of the non-symmetric property spike pulse, but the physical quantities of the frequency, voltage, and so on, corresponding to the physiological mechanism should be set accurately, and in particular, a dynamic transition of the non-symmetric property spike pulse waveform comes out to be a very important element having a large influence to the therapeutic effect.

### SUMMARY OF THE INVENTION

An object of the present invention is to provide a low frequency therapy apparatus in which physical quantities of frequency, voltage, and so on, corresponding to a physiological mechanism of human body is set more accurately, and in addition, it makes an optimum setting for a dynamic transition of a waveform, so that a therapeutic effect to soothe or remove a pain can be more increased.

In addition, another object of the present invention is to provide the low frequency therapy apparatus which can be implemented easily without requiring major additional parts or a structural modification in particular, and can contribute to reduce the cost and size thereof, compared to a conventional low frequency therapy apparatus.

To attain these objects, the present invention, constituting a low frequency therapy apparatus includes: a spike pulse generating circuit portion, outputting a non-symmetric property spike pulse from a secondary winding of an output transformer; a grip portion to be held by a hand; a positive electrode portion having a plurality of pin conductors, provided at a tip portion side of the grip portion being an electrode of one side to which the non-symmetric property spike pulse outputted from the spike pulse generating circuit portion is supplied; and a negative electrode portion, being an electrode of the other side to which the corresponding non-symmetric property spike pulse is supplied, and wherein the non-symmetric property spike pulse is set to have a frequency of 8.0±0.5 Hz, a pulse width of a range from 210 μsec to 650 μsec, and wherein a turns ratio between a primary winding and the secondary winding of the output transformer is set so that a positive peak voltage value of the non-symmetric property spike pulse during the period of the pulse width is to be from 60 V to 100 V, when being applied at 300 KΩ load which corresponds to a load resistance of a human body, and a positive voltage during the period of the pulse width is kept to be equal to or more than a predetermined voltage value.

### BRIEF DESCRIPTION OF THE DRAWINGS

FIG. 1A, FIG. 1B, and FIG. 1C are waveform charts of signals at the respective portions of a low frequency therapy apparatus according to a preferred embodiment of the present invention;

FIG. 2 is a block circuit diagram of the low frequency therapy apparatus;

FIG. 3 is an external perspective view seen from a front side of the low frequency therapy apparatus;

FIG. 4 is an external perspective view seen from a rear side showing a part of the low frequency therapy apparatus;

FIG. 5 is an explanatory view of how to use the low frequency therapy apparatus; and

FIG. 6A and FIG. 6B are action explanatory views of the low frequency therapy apparatus.

### DETAILED DESCRIPTION OF THE PREFERRED EMBODIMENT(S)

Next, a preferable embodiment relating to the present invention will be cited and explained in detail with reference to the drawings. It should be noted that the attached drawings are not for specifying the present invention but to facilitate understanding of the present invention. As for well-known portions, in order to avoid making the present invention obscure, detailed explanation will be restrained.

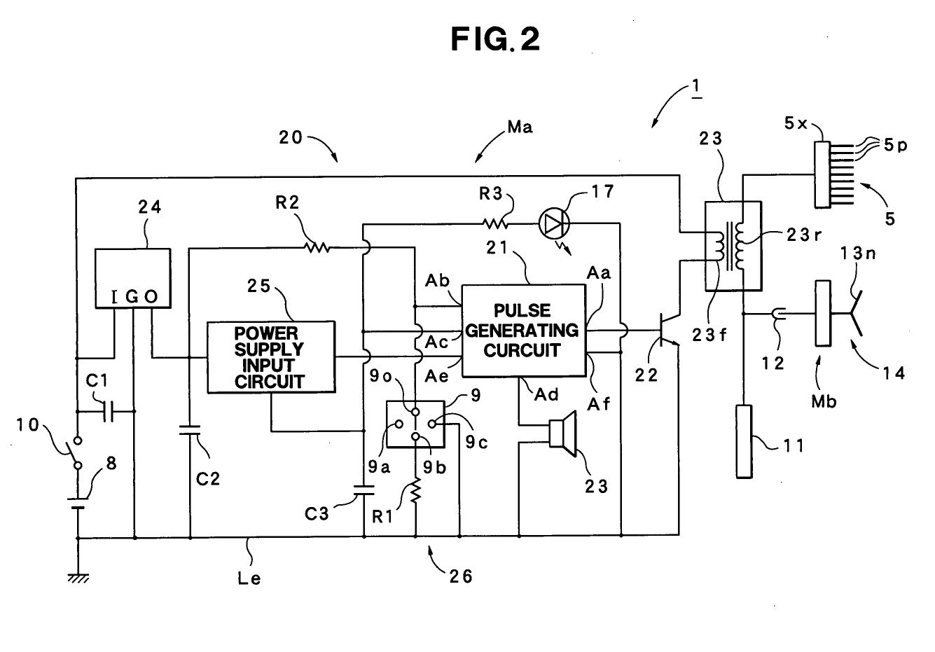

First, a configuration of a low frequency therapy apparatus 1 according to the present embodiment will be explained with reference to FIG. 2 to FIG. 4.

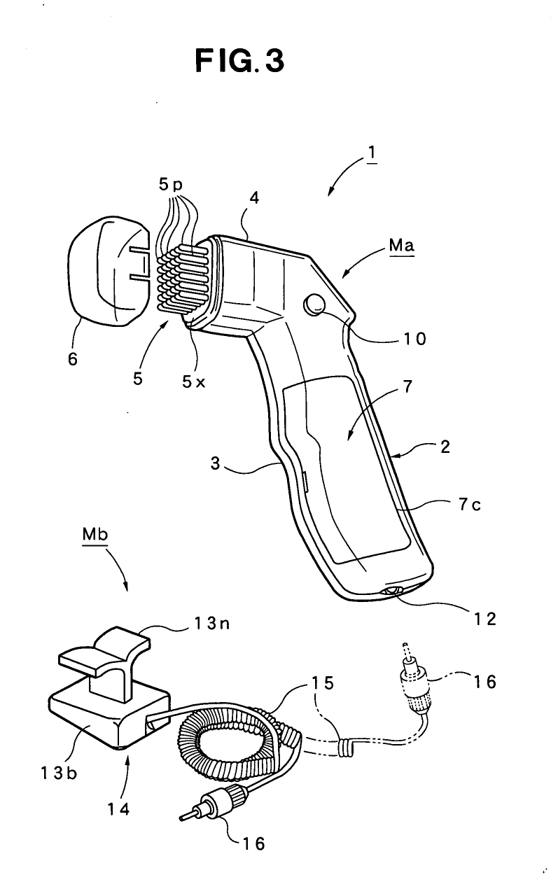

The low frequency therapy apparatus 1 includes a therapy apparatus main body Ma and an electrode unit Mb, as shown in FIG. 3. The therapy apparatus main body Ma has a housing 2, one side of the housing 2 is a grip portion 3 so as to be held by a hand Hc, and the other side of the housing 2 is a positive electrode mounting portion 4 to mount a positive electrode portion 5 at the tip portion thereof. The positive electrode mounting portion 4 projects at approximately an angle of 110 degrees relative to the grip portion 3, and a pin mounting disk portion 5x is fixed at the tip portion thereof perpendicular to the projecting direction. On the pin mounting disk portion 5x, there are a plurality of conductive pins 5p perpendicularly projected. Hereby the positive electrode portion 5 being an electrode of one side to which a later-described non-symmetric property spike pulse Ps is supplied, is constituted.

The respective conductive pins 5p are made of a conductive material such as a stainless steel material or the like, the diameter is selected to be 2 mm, and the tip portion thereof is formed to be spherical. Besides, the spacing between the neighbor conductive pins 5p is set to be 2 mm. The diameter of the conductive pins 5p is selected to have the range from 1 mm to 3 mm, thereby it is optimum from the point of view of usability, and the spacing between the neighbor conductive pins 5p is selected to have the range from 1 mm to 2 mm, thereby it is advantageous from the point of view of assuring the spacing which does not miss the pain spot in the affected part. In case of the shown example, the length of the respective conductive pins 5p is selected to be 12 mm, but there is no restriction if the length is suitable for use the low frequency therapy apparatus 1. Further, the number of the respective conductive pins 5p is selected to be 36, but there is no restriction in the number of pins if the current can be effectively flowed to the affected part having the pain. Incidentally, reference numeral 6 denotes a cap which is attached/detached to/from the tip portion of the positive electrode mounting portion 4, to cover and protect the respective conductive pins 5p when it is placed thereon.

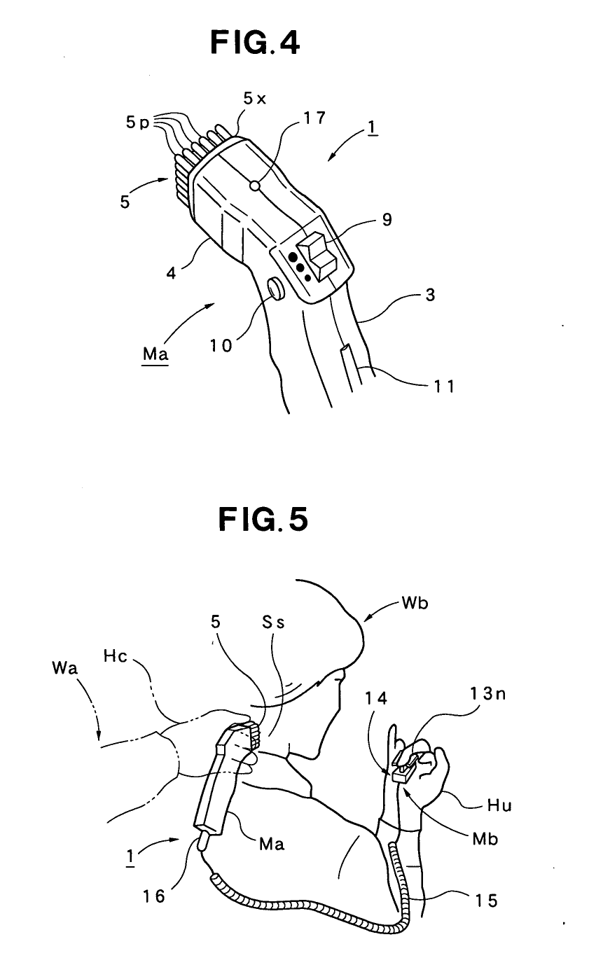

At a specified position of the grip portion 3, a battery accommodating portion 7 having an opening and closing cover 7c, is provided, and a battery 8 (FIG. 2) of 9 V is accommodated in the battery accommodating portion 7. Besides, on the back surface of the grip portion 3, a strong and weak changing over switch 9 is arranged as shown in FIG. 4, and on the side surface of the grip portion 3, an activation switch 10 is arranged. Meanwhile, on the back surface (outside surface) of the grip portion 3, an electrode plate (electrode member) 11 formed by a conductive material such as a stainless steel material or the like, which can be in contact with a hand Hc, when it is held by the corresponding hand Hc, is attached. The electrode plate 11 composes a negative electrode portion being the other electrode to which the later-described non-symmetric property spike pulse Ps is supplied. On the end face portion of the grip portion 3, a connection jack 12 to connect the electrode unit Mb, is arranged.

On the other hand, the electrode unit Mb includes an unit main body 14 composed of an electrode portion 13n formed by a conductive material such as a stainless steel material or the like, to put on and bring into contact with a finger of a hand Hu, and a base portion 13b to support the electrode portion 13n, and a code 15 of which one end is connected to the electrode portion 13n and the other end is connected to a connection pin 16. The connection pin 16 attaches/detaches to/from the connection jack 12 provided at the therapy apparatus main body Ma. Incidentally, reference numeral 17 denotes a LED (activation lamp) which lights up when the activation switch 10 is turned on (at the time of power-on).

Further, the grip portion 3 has a spike pulse generating circuit portion 20 which is built therein. FIG. 2 shows a block circuit of the spike pulse generating circuit portion 20. The spike pulse generating circuit portion 20 includes a pulse generating circuit 21, an output transistor 22, an output transformer 23, a power regulator 24, a power supply input circuit 25, a strong and weak changing over circuit 26, and so on, as main circuits and parts.

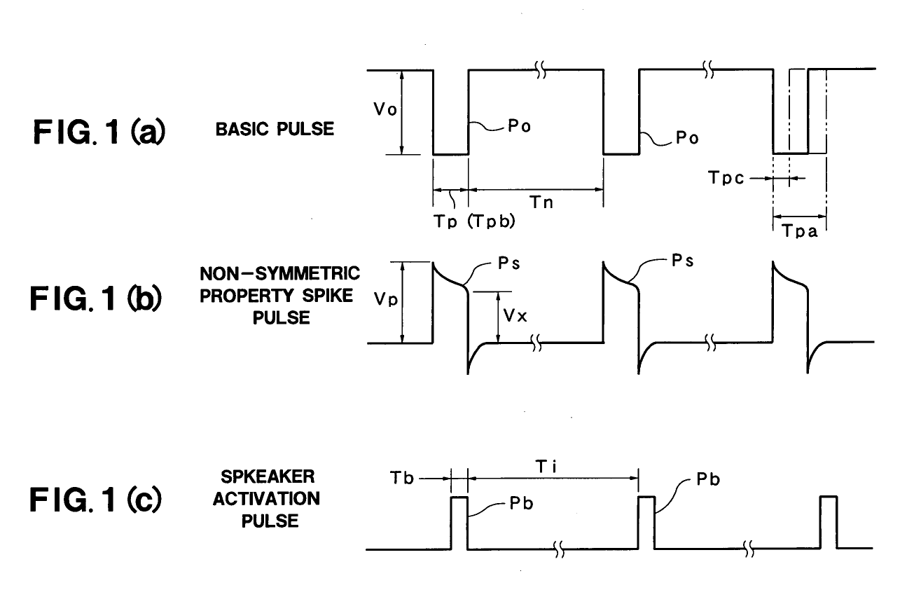

In this case, the pulse generating circuit 21 is an IC chip generating and outputting a basic pulse (pulse signal) Po shown in FIG. 1A, and having six ports. A first port Aa of the pulse generating circuit 21 is an output port to output the basic pulse Po. The basic pulse Po is set to have a frequency of 8.0 Hz, a pulse voltage Vo of −5 V, and a pulse width Tp of a range from 210 μsec to 650 μsec. The optimum frequency is 8.0 Hz, but the range of 8.0±0.5 Hz is fully acceptable.

Besides, by changing the pulse width Tp, the strong and weak level of the stimulus (energy) can be changed over. The strong and weak changing over circuit 26 is a circuit to change over the strong and weak level of the stimulus by changing the pulse width Tp, and includes the above-described strong and weak changing over switch 9. In this case, a movable contact 9o of the strong and weak changing over switch 9 is connected to a second port Ab of the pulse generating circuit 21, and also, a first fixed contact 9a is set to be no connection, a second fixed contact 9b is connected to a ground line Le via a resistor R1, and a third fixed contact 9c is directly connected to the ground line Le. Hereby, when the strong and weak changing over switch 9 is changed over to the first fixed contact 9a, the second port Ab can be supplied the power supply voltage via a resistor R2, thereby the pulse width Tp becomes 650 μsec as shown by Tpa in FIG. 1A, and the stimulus becomes “strong”. Further, when the strong and weak changing over switch 9 is changed over to the second fixed contact 9b, the second port Ab can be supplied at a divided power supply voltage, thereby the pulse width Tp becomes 480 μsec as shown by Tpb in FIG. 1A, and the stimulus becomes “middle”. Furthermore, when the strong and weak changing over switch 9 is changed over to the third fixed contact 9c, the voltage supplied to the second port Ab becomes “0 (zero)”, thereby the pulse width Tp becomes 210 μsec as shown by Tpc in FIG. 1A, and the stimulus becomes “weak”. Incidentally, a no-pulse period Tn becomes approximately 125 msec by the above-described setting of the pulse width Tp, and the frequency setting of 8.0 Hz.

Meanwhile, the first port (output port) Aa of the pulse generating circuit 21 is connected to one end of a primary winding 23f of the output transformer 23 via the output transistor 22. And, the other end of the primary winding 23f is connected to a positive electrode of the battery 8 via the activation switch 10. Incidentally, a negative electrode of the battery 8 is connected to the ground line Le. In this case, a turns ratio between the primary winding 23f and a secondary winding 23r of the output transformer 23 is set to be “primary winding:secondary winding=1:18”. This turns ratio is an important setting element to obtain a desirable non-symmetric property spike pulse Ps, and the turns ratio is set to be “1:18” as described above, thereby a good non-symmetric property spike pulse Ps can be obtained at the secondary winding 23r of the output transformer 23.

Accordingly, when the load resistance of 300 kΩ which corresponds to the load resistance of the human body is connected, the non-symmetric property spike pulse Ps having an optimum waveform shape as shown in FIG. 1B can be obtained. In this case, a positive peak voltage value Vp during the period of the pulse width Tp of the obtained non-symmetric property spike pulse Ps becomes from 60 V to 100 V, and such a waveform that a positive voltage during the period of the pulse width Tp keeps equal to or more than the predetermined voltage value Vx can be obtained. Incidentally, it is most preferable that the turns ratio of the output transformer 23 is set to be “primary winding:secondary winding=1:18”, but the range from “1:14” to “1:22” is also acceptable.

Besides, one end of the secondary winding 23r of the output transformer 23 is connected to a positive electrode portion 5 (the respective conductive pins 5p), and the other end of the secondary winding 23r is connected to the electrode plate 11 composing the negative electrode portion, and the connection jack 12, respectively.

Meanwhile, an input terminal of the power regulator 24 is connected to the positive electrode of the battery 8 via the activation switch 10, and an output terminal thereof is connected to the input side of the power supply input circuit 25. Further, the output side of the power supply input circuit 25 is connected to the third port (power port) Ac of the pulse generating circuit 21, and also, a series circuit of a resistor R3 and the LED 17 is connected between the third port Ac and the ground line Le. Further, between a fourth port Ad of the pulse generating circuit 21 and the ground line Le, a speaker 28 is connected, and also, a fifth port Ae of the pulse generating circuit 21 and the power supply input circuit 25 are connected. Incidentally, a sixth port (ground port) Af of the pulse generating circuit 21 is connected to the ground line Le. In addition, reference symbols C1, C2, and C3 in the spike pulse generating circuit portion 20 denote condensers to remove noises and so on. The low frequency therapy apparatus 1 configured as above-described can be implemented easily without requiring major additional parts or a structural modification in particular, and can be contributed to reduce the cost and size thereof, compared to the conventional low frequency therapy apparatus.

Next, the operation and the way how to use the low frequency therapy apparatus 1 having such a configuration, will be explained with reference to FIG. 1 to FIG. 6.

First, a case when a therapy performer Wa performs a therapy to another therapy receiver Wb by using the low frequency therapy apparatus 1 as shown in FIG. 5, is explained. In this case, the connection pin 16 of the electrode unit Mb is plugged into the connection jack 12 of the therapy apparatus main body Ma, so as to connect them. The electrode portion 13n of the electrode unit Mb is therefore connected to the secondary winding 23r of the output transformer 23. And as shown in FIG. 5, the unit main body 14 of the electrode unit Mb is placed on a hand Hu of the therapy receiver Wb, and a finger of the hand Hu is placed on the electrode portion 13n so as to bring into contact with each other.

Meanwhile, the strong and weak changing over switch 9 is changed over, thereby the mode of the “strong”, the “middle”, or the “weak” is selected. In this case, for example, the mode of the “strong” or the “middle” is selected for a therapy of a human body region from the neck to the bottom, and the mode of the “weak” can be selected for the therapy of the human body region from the neck to the head. In addition, if necessary, the mode can be selected according to the degree of the pain and so on in the affected part being the object of the therapy.

The therapy performer Wa holds the grip portion 3 of the therapy apparatus main body Ma by the hand Hc, and dabs the tip portions of the conductive pins 5p at the skin surface Ss corresponding to the affected part of the therapy receiver Wb having the pain. The tip portions of the conductive pins 5p are dabbed at the skin surface Ss, thereafter the activation switch 10 is pressed to turn on the apparatus. Hereby, a power dispatching is performed to the pulse generating circuit 21, and the basic pulse (pulse signal) Po shown in FIG. 1A is outputted from the first port (output port) Aa of the pulse generating circuit 21. The basic pulse Po is amplified by the output transistor 22, and is supplied to the primary winding 23f of the output transformer 23.

The conductive pins 5p are in contact with the skin surface Ss of the affected part, and the electrode portion 13n is in contact with the hand Hu, thereby a load resistance of the human body is connected between the both ends of the secondary winding 23r of the output transformer 23. Generally, the load resistance of the human body is substantially 300 KΩ, and it means that the load resistance of substantially 300 KΩ is connected between the both ends of the secondary winding 23r. As a result, the non-symmetric property spike pulse Ps shown in FIG. 1B is applied to the skin surface Ss of the affected part, from the conductive pins 5p. In this case, the non-symmetric property spike pulse Ps has the positive peak voltage value Vp during the period of a pulse width Tp of a range from 60 V to 100 V, and the positive voltage during the period of the pulse width Tp keeps equal to or more than a predetermined voltage value Vx. The waveform of the non-symmetric property spike pulse Ps shown in FIG. 1B represents the case when the “middle” mode is selected, and the pulse width Tp is 480 μsec. The positive peak voltage value Vp at this time becomes 80 V and the predetermined voltage value Vx keeps equal to or more than the half value (40 V) of the positive peak voltage value Vp. The waveform shape of the non-symmetric property spike pulse Ps as above-described is important, and particularly, this waveform shape can be obtained by setting the turns ratio between the primary winding 23f and the secondary winding 23r of the output transformer 23 to be above-described 1:18. If such a waveform shape cannot be obtained, for example, when the positive peak voltage value Vp becomes too high, the sufficient therapeutic effect cannot be obtained. Incidentally, when the pulse width Tp is within the range from 210 μsec to 650 μsec, the condition that the positive peak voltage value Vp during the period of the pulse width Tp becomes from 60 V to 100 V, and the positive voltage during the period of the pulse width Tp keeps equal to or more than the predetermined voltage value Vx (the half value of the positive peak voltage value Vp), is secured.

On the other hand, the activation switch 10 is turned on, thereby the LED (activation lamp) 17 lights up. Besides, a buzzer signal shown in FIG. 1C is outputted from the fourth port Ad of the pulse generating circuit 21, by means of a timer function built in the pulse generating circuit 21. The buzzer signal is outputted by an output pulse Pb which is set to have the voltage of 5 V, the pulse width Tb of approximately 1 sec, at every time interval of Ti. By the output of the output pulse Pb, the buzzer sound is ringed from the speaker 28 at every time interval of Ti. Therefore, the buzzer sound can be an indicator to change the position of the skin surface Ss to be dabbed the conductive pins 5p (positive electrode portion 5), thereby the therapy can be performed in just proportion, and the problem to perform the therapy at one place for unnecessarily long time can be avoided. Incidentally, the time Ti can be set randomly, but in general, 60 sec is desirable.

By using the low frequency therapy apparatus 1 in such a manner, a physical quantity of the frequency, the voltage, and so on, corresponding particularly to the physiological mechanism of the human body can be set more accurately, and also, the optimum setting is performed for the dynamic transition of the waveform, thereby the therapeutic effect to soothe and remove the pain can be more increased.

By the way, the therapeutic action by using the low frequency therapy apparatus 1 is considered to come from the following mechanism. As above-described, a pain passes from peripheral nerves through a spinal cord being a central nerve, reaches a cerebrum and makes it aware of a pain, by a stimulus being a cause of the pain. At this time, when the stimulus is transferred on the level of spinal cord, a motor nerve is excited and a muscle tension is increased. Furthermore, a sympathetic nerve is activated, and a substance called catecholamine is secreted from an adrenal gland. Next, blood vessels shrink and a blood shortage comes out in cellular structures. When this abnormal metabolic turnover continues, a bradykinin or a serotonine known as pain producing substances are secreted, and those substances pass from the peripheral nerves through the spinal cord, the pain is recognized at the cerebrum again.

Meanwhile, the human body adjusts the balance by the activity of nerves and electrical signals of ion. Concretely speaking, there exists more plus electric charge inside of cells, and more minus electric charge on the cell membrane outside of cells, and a good health is maintained by the even ion balance. Generally, the ion balance is maintained by potassium ions and sodium ions, and there are more potassium ions inside of the cell membrane, on the contrary, there are less potassium ions and more sodium ions outside of the cell membrane, and they are adjusted by a cerebrum and information on end of nerve cells.

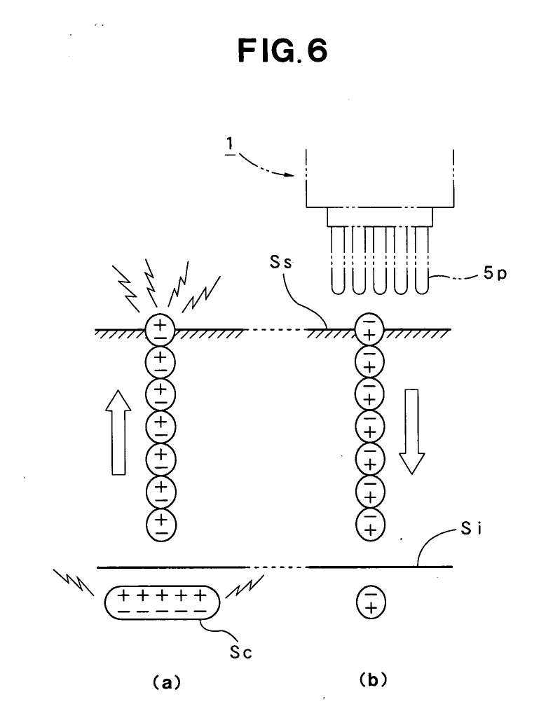

On the other hand, when an organ is damaged caused by disease or the like, the number of potassium ions increase, and a plus ion group is formed. And minus ions existing in cells or in the nerve cells of the peripheral tissues are attracted. Thereby, only the plus ions remain in the peripheral tissues of the opposite side. Such an unbalance status of ions are transferred from cells to cells in turn, and transferred from visceral organs to skin cells, from central nerves to a cerebrum in nerve cells. That is to say, the pain is what is called a reversed phenomenon of ions, in which the plus ions become advantageous because more potassium ions exist outside of cells though it should be inside of cells under normal circumstances. The conceptual view of the above-described status is shown in FIG. 6A. In the corresponding view, reference symbol Ss denotes a skin surface, reference symbol Si denotes a visceral organ wall, and reference symbol Sc denotes a diseased cell, respectively.

It is considered that the low frequency therapy apparatus 1 according to the present embodiment focuses attention on a transfer recognition mechanism of pain and the non-symmetric property spike pulse Ps of low frequency is intermittently applied to the affected part of the human body having a pain, and the current based on the non-symmetric property spike pulse Ps is flowed to the human body, thereby the reversed state of ions are turned back to the normal status. As a result, the pain cycle of the affected part is cut off, thereby the pain is soothed and removed in a short time. To speak more concretely, the ischemic condition of a body tissue is abolished, a production of a new pain producing substance is suppressed, and an abnormal low frequency which comes out of a pain-sensing organ is adjusted, thereby a pain caused by muscle ache or the like is soothed and removed. The conceptual view of a normal status of ions is shown in FIG; 6B.

Hereinabove, the case when the low frequency therapy apparatus 1 is used and a therapy performer Wa performs a therapy to another therapy receiver Wb, is explained, but this low frequency therapy apparatus 1 can be used to perform a therapy for oneself. In this case, the connection pin 16 of the electrode unit Mb is pulled out and detached from the connection jack 12 of the therapy apparatus main body Ma.

Then the one holds the grip portion 3 by ones own hand Hc, and dabs the tip portions of the conductive pins 5p to the skin surface of the affected part having a pain. At this time, the one keeps in mind that the hand Hc of ones own is in contact with the electrode plate 11 attached on the back surface (outside surface) of the grip portion 3. The tip portions of the conductive pins 5p are dabbed at the skin surface, thereafter the activation switch 10 is pressed to turn on the apparatus. Furthermore, one can easily perform the same therapy as the case when the therapy performer Wa performs a therapy to another therapy receiver Wb as described above, for oneself.

Hereinabove, the present embodiment has been explained in detail. However, the present invention is not limited to these embodiments, it should be considered to be acceptable to modify any detail of configuration or method of implementation without departing from the spirit of the present invention, and at the same time to add or cancel some details as necessary. For example, the pulse width Tp of the non-symmetric property spike pulse Ps can be set so as to be continuously changeable between the range from 210 μsec to 650 μsec. Furthermore, the case is shown that the spike pulse generating circuit portion 20 is built in the grip portion 3, but it is not necessarily to be built in the grip portion 3.

What is claimed is:

1. A low frequency therapy apparatus, comprising:
a spike pulse generating circuit portion, outputting a non-symmetric property spike pulse from a secondary winding of an output transformer;
a grip portion to be held by a hand;
a positive electrode portion, provided at a tip portion side of said grip portion and having a plurality of conductive pins being an electrode of one side to which the non-symmetric property spike pulse outputted from said spike pulse generating circuit portion is supplied; and
a negative electrode portion, being an electrode of the other side to which the corresponding non-symmetric property spike pulse is supplied, and
wherein a non-symmetric property spike pulse is set to have a frequency of 8.0±0.5 Hz, a pulse width of a range from 210 μsec to 650 μsec, and
wherein a turns ratio between a primary winding and a secondary winding of the output transformer is set so that a positive peak voltage value of the non-symmetric property spike pulse during the period of the pulse width is to be from 60 V to 100 V, when being applied at 300 KΩ load which corresponds to a load resistance of a human body, and a positive voltage during the period of the pulse width is kept to be equal to or more than a predetermined voltage value.

2. The low frequency therapy apparatus according to claim 1, wherein the conductive pin is set to have a diameter of a range from 1 mm to 3 mm, and a spacing between the neighbor conductive pins of a range from 1 mm to 2 mm.

3. The low frequency therapy apparatus according to claim 1, further comprising,
a setting change circuit, in which the pulse width of the non-symmetric property spike pulse is changeable within the range from 210 μsec to 650 μsec.

4. The low frequency therapy apparatus according to claim 1, wherein the turns ratio of the output transformer is set to be “the primary winding:the secondary winding” is from “1:14” to “1:22”.

5. The low frequency therapy apparatus according to claim 1, wherein said spike pulse generating circuit portion is built in said grip portion.

6. The low frequency therapy apparatus according to claim 1, wherein said negative electrode portion is arranged on the outside surface of said grip portion, and is an electrode member which is contactable with a hand when being held by the corresponding hand.

7. The low frequency therapy apparatus according to claim 1, wherein said negative electrode portion is constituted to be derivable from said grip portion via a connection code, and is an electrode unit which is contactable with a human body.
